(19) United States
(12) Patent Application Publication
(10) Pub. No.: US 2006/0036308 A1
(43) Pub. Date: Feb. 16, 2006

(54) Stent with extruded covering

(75) Inventor: Justin Goshgarian, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 10/917,594

(22) Filed: Aug. 12, 2004

(51) Int. Cl. A61F 2/06

(52) U.S. Cl. 623/1.11; 623/1.13; 623/1.16; 427/2.25; 264/279; 425/111; 425/113

(57) ABSTRACT

The stent having an extruded covering of the present invention, and method of making the same, provides a stent 112 having stent elements 116 forming cells 118, and a covering 114 disposed on the stent 112. The seamless covering 114 encloses the stent elements 116 and the cells 118. The stent having an extruded covering is fabricated by compressing a stent assembly radially 252, applying molten polymer to the stent elements and cells 254, expanding the stent assembly radially to form a covering 252, and cooling the stent assembly 254. The stent assembly can then be separated into individual stents. The compression can be performed by drawing the stent assembly through a contracting die 154 and the molten polymer can be applied in an extruder 156. Before or after separating the stent assembly into individual stents, post treatment can be performed or coatings applied.

### TECHNICAL FIELD

The technical field of this disclosure is medical implant devices, particularly, stents having an extruded covering and methods of making the same.

### BACKGROUND OF THE INVENTION

Stents are generally cylindrical shaped devices that are radially expandable to hold open a segment of a blood vessel or other anatomical lumen after implantation into the body lumen. Typical uses include stent grafts to shunt blood through aortic aneurysms and angioplasty stents to dilate stenotic blood vessels. Stents have been developed with coatings to deliver drugs or other therapeutic agents.

Aneurysms can occur in any number of blood vessels, but are of particular concern in the abdominal aorta and thoracic aorta. Abdominal aortic aneurysms represent one of the most common types of aneurysms and result in about 15,000 deaths annually in the United States. An aneurysm is produced when a thinning or weak spot in a vessel wall dilates eventually posing a health risk from its potential to rupture, clot, or dissect. An aneurysm frequently occurs in arteries, but may also form in veins. The etiology of aneurysm formation is not entirely understood, but is thought to be related to congenital thinning of the artery, atherosclerotic vessel degeneration, vessel trauma, infection, smoking, high blood pressure, and other causes leading to vessel degeneration. Left untreated, abdominal aortic aneurysms may lead to gradual vessel expansion, thrombus formation leading to stroke or other vessel blockage, vessel rupture, shock, and eventual death.

Abdominal aortic aneurysms are generally localized on long abdominal aortic sections below the renal arteries and oftentimes extend into one or both of the iliac arteries. The aneurysm may begin with a small vessel distension that progressively enlarges at a variable and unpredictable rate. An abdominal aortic aneurysm may enlarge at an average rate of about. 0.3-0.5 cm per year. The abdominal aortic aneurysm may continue to enlarge in a silent fashion until a catastrophic event, such as a rupture, occurs. The best predictor of rupture risk is size: rupture is relatively uncommon in abdominal aortic aneurysm less than 5 cm. Once reaching about 8 cm, however, there is about a 75 percent chance of rupture within a year. Besides rupture, another risk of abdominal aortic aneurysms is thrombus dissection. As the vessel enlarges, a thrombus may develop in the aneurysm due to perturbations in blood flow dynamics. Pieces of the clot may eventually loosen and carry away, eventually forming blockages in the legs, lungs, or brain.

Abdominal aortic aneurysms are most commonly treated in open surgical procedures, where the diseased vessel segment is bypassed and repaired with an artificial vascular graft. While considered an effective surgical technique, particularly considering the alternative of the usually fatal ruptured abdominal aortic aneurysm, conventional vascular graft surgery suffers from a number of disadvantages. The surgical procedure is complex and requires experienced surgeons and well equipped surgical facilities. Even with the best surgeons and equipment, patients suffering from such aneurysms are often elderly and weakened from cardiovascular and other diseases. This factor reduces the number of patients eligible for surgery. Even for eligible patients prior to rupture, conventional aneurysm repair has a relatively high mortality rate, usually from 2 to 10%. Morbidity related to the conventional surgery includes myocardial infarction, renal failure, impotence, paralysis, and other conditions. Even with successful surgery, recovery takes several weeks and often requires a lengthy hospital stay.

To overcome some of the drawbacks associated with open surgery, a variety of endovascular prosthesis placement techniques have been proposed. Without the need for open abdominal surgery, patient complications and recovery time may be significantly reduced. One endovascular abdominal aortic aneurysm repair technique involves a tubular prosthesis deployed by remote insertion through a femoral artery. The prosthesis may include a synthetic graft sheath body supported by an expandable structure such as a stent. The stent may be self-expanding or balloon-expanding and typically includes means for anchoring the prosthesis to the vessel wall. The stent graft prosthesis acts as a shunt to carry blood flow from a healthy portion of the aorta, through the aneurysm, and into one or both of the iliac artery branches. The prosthesis excludes any thrombus present in the aneurysm while providing mechanical reinforcement of the weakened vessel reducing the risk of dissection and rupture, respectively.

The stent graft design presents problems in fabrication and use. Each stent graft is manufactured individually. The graft fabric is attached to each stent by hand in a slow, labor-intensive, expensive process. Sewing the graft to the stent is laborious. Heat laminating the graft to the stent is quicker, but requires thick graft material, which may not bond well to the stent. In use, both the sewn and laminated designs have drawbacks. The needle hole perforations in a sewn stent may allow leakage through the graft material. Sagging graft material may provide sites for thrombus formation. Laminated stent grafts may also sag, but can also form aneurysms in the graft material. The thicker graft material can also allow thrombus formation at the stent graft ends, where the high profile of the stent graft projects into the blood vessel.

Stents are used in other medical therapeutic applications, including intravascular angioplasty. For example, a balloon catheter device is inflated during PTCA (percutaneous transluminal coronary angioplasty) to dilate a stenotic blood vessel. The stenosis may be the result of a lesion such as a plaque or thrombus. After inflation, the pressurized balloon exerts a compressive force on the lesion thereby increasing the inner diameter of the affected vessel. The increased interior vessel diameter facilitates improved blood flow. Soon after the procedure, however, a significant proportion of treated vessels re-narrow.

Short flexible cylindrical stents, constructed of metal or various polymers are implanted within the vessel to maintain lumen size to prevent restenosis. The stents acts as a scaffold to support the lumen in an open position. Various configurations of stents include a cylindrical tube defined by a mesh, interconnected stents or like segments. Some exemplary stents are disclosed in U.S. Pat. No. 5,292,331 to Boneau, U.S. Pat. No. 6,090,127 to Globerman, U.S. Pat. No. 5,133,732 to Wiktor, U.S. Pat. No. 4,739,762 to Palmaz and U.S. Pat. No. 5,421,955 to Lau. Balloon-expandable stents are mounted on a collapsed balloon at a diameter smaller than when the stents are deployed. Stents can also be self-expanding, growing to a final diameter when deployed without mechanical assistance from a balloon or like device.

Stents have been used with coatings to deliver drug or other therapy at the site of the stent. The coating can be applied as a liquid containing the drug or other therapeutic agent dispersed in a polymer/solvent mixture. The liquid coating then dries to a solid coating upon the stent. The liquid coating can be applied by dipping or spraying the stent while spinning or shaking the stent to achieve a uniform coating. Combinations of the various application techniques can also be used.

The purpose of the coating is to provide the drug to the tissue adjacent to the stent, such as the interior wall of an artery or vessel. Typically, the coating is applied as one or more layers over the stent wires. Some coatings containing drugs biodegrade over six months or more to deliver the drugs.

U.S. Pat. No. 6,139,573 to Sogard et al. discloses a method and apparatus for forming a covered endoprosthesis employing a conformed polymeric coating about an expandable stent. A first polymeric liner is positioned about an inner surface of the tubular stent and a second polymeric liner is positioned about an outer surface of the tubular stent. The first and second polymeric liners are conformed to the tubular stent and laminated together through the open construction of the stent at a location coextensive with the inner surface of the tubular stent.

U.S. Pat. No. 6,214,039 to Banas et al. discloses a radially expandable endoluminal covered stent assembly and a method and apparatus for making the same. A longitudinally and radially expanded polytetrafluoroethylene tubular graft is circumferentially engaged about one or more radially expandable stents and is retained thereon by a radial recoil force exerted by the tubular graft against the stent.

U.S. Pat. No. 6,296,661 and 6,245,100 to Davila et al. disclose a stent-graft and method of making a stent-graft for insertion into target site within a vessel of a patient. The method uses a self-expanding tubular elastic outer stent having a crimped and expanded state, a tubular flexible porous graft member inserted along an interior of the outer stent, and a self-expanding tubular elastic inner stent inserted along an interior of the graft member. The graft member has front and back ends which are folded over and bonded to the front and back ends of the outer stent to form cuffs.

U.S. Pat. No 6,270,523 to Herweck et al. discloses a radially expandable support body enveloped within a cocoon. In a preferred construction, the support is a stent, and a tube of polymeric material, e.g., polytetraeluoroethylene (PTFE), passes through the interior of the stent body and is turned back upon itself over the stent to form a cuff. The assembly is then heated and the outer layer contacts and coalesces with the inner layer, closely surrounding the stent body within a folded envelope having a continuous and seamless end.

U.S. Pat. No 6,395,212 to Solem discloses a method of making a covered stent introducing a stent into a tube of a film material and exposing the tube to an elevated temperature to reduce the diameter of the tube, such that the stent is affixed within the tube. Collars may be formed at the ends of the tube and may also be covered by the film material.

It would be desirable to have a stent having an extruded covering and method of making the same that would overcome the above disadvantages.

### SUMMARY OF THE INVENTION

One aspect of the present invention provides a stent having an extruded covering with a seamless, easy to apply covering completely enclosing the stent.

Another aspect of the present invention provides a stent having an extruded covering with the covering intimately connected to the stent.

Another aspect of the present invention provides a stent having an extruded covering with a thin, taut covering without perforations.

Another aspect of the present invention provides a stent having an extruded covering with a low profile.

Another aspect of the present invention provides a stent having an extruded covering, which allows several stents to be manufactured in a batch.

Another aspect of the present invention provides a stent having an extruded covering, which can be manufactured without laborious, time-consuming, expensive hand labor.

The foregoing and other features and advantages of the invention will become further apparent from the following detailed description of the presently preferred embodiments, read in conjunction with the accompanying drawings. The detailed description and drawings are merely illustrative of the invention, rather than limiting the scope of the invention being defined by the appended claims and equivalents thereof.

### BRIEF DESCRIPTION OF THE DRAWINGS

FIG. 1 shows a stent delivery system made in accordance with the present invention with the stent partially deployed.

FIGS. 2 & 3 show a stent and a cross section, respectively, of a stent with an extruded covering made in accordance with the present invention.

FIG. 4 shows a stent assembly for use in a method of manufacturing a stent with an extruded covering made in accordance with the present invention.

FIGS. 5 & 6 show a method of manufacturing a stent with an extruded covering made in accordance with the present invention.

FIG. 7 shows another method of manufacturing a stent with an extruded covering made in accordance with the present invention.

FIG. 8 shows yet another method of manufacturing a stent with an extruded covering made in accordance with the present invention.

FIG. 9 shows a flow chart of a method of manufacturing a stent with an extruded covering made in accordance with the present invention.

### DETAILED DESCRIPTION OF THE PRESENTLY PREFERRED EMBODIMENT

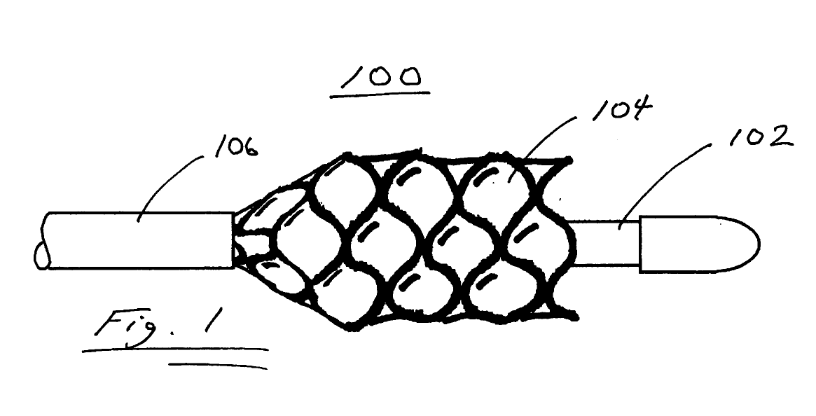

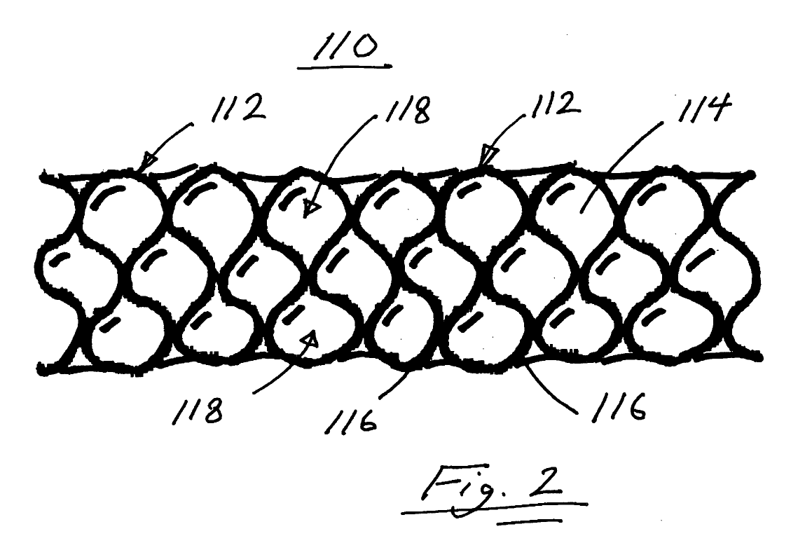

FIG. 1 shows a stent delivery system made in accordance with the present invention with the stent partially deployed. The stent delivery system 100 includes a catheter 102, an extruded stent 104 disposed on the catheter 102, and a sheath 106 slidably disposed about the stent 104. The extruded stent 104 can be self-expanding, so that the extruded stent 104 is compressed within the sheath 106 for delivery to the implantation site and the sheath 106 is retracted to allow the extruded stent 104 to expand for implantation. The extruded stent 104 is shown partially deployed as the sheath 106 is being retracted, so that the distal end of the extruded stent 104 is expanded. In another embodiment, the catheter 102 can be a balloon catheter, such as a balloon catheter used for PTCA (percutaneous transluminal coronary angioplasty). The extruded stent 104 can be disposed about the balloon and the balloon inflated to expand the extruded stent 104. Balloons may be manufactured from a material such as polyethylene, polyethylene terephthalate (PET), nylon, Pebax® polyether-block co-polyamide polymers, or the like. A sheath may not be required to restrain the extruded stent if the extruded stent is not self-expanding, although a sheath can be used to retain the extruded stent on the balloon.

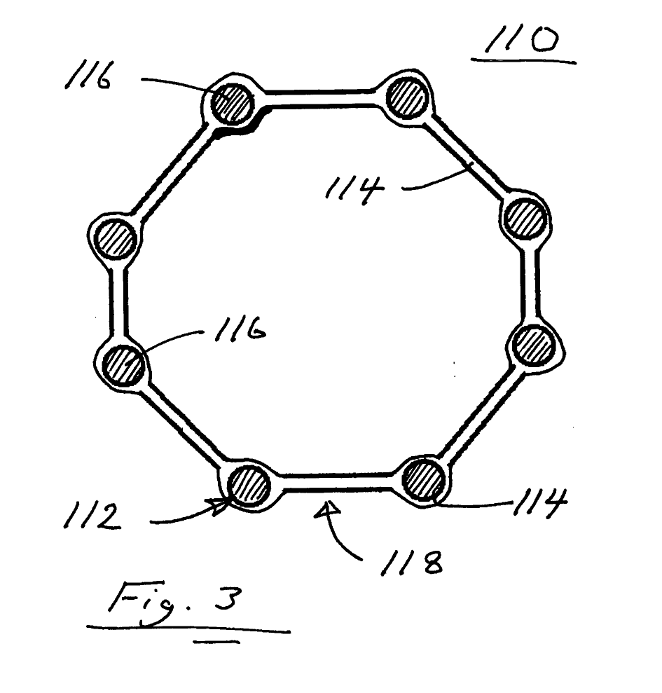

FIGS. 2 & 3, in which like elements share like reference numbers, show a stent and a cross section, respectively, of a stent with an extruded covering made in accordance with the present invention. The extruded stent 110 comprises a stent 112 and a covering 114 disposed on the stent 112. The stent 112 has stent elements 116 forming cells 118. The covering 114 encloses the stent elements 116 and the cells 118. In another embodiment, additional coatings can be applied to the covering 114. In one example, a lubricious coating can be applied to the outer diameter of the covering 114 to improve deployment of a self-expanding extruded stent from the sheath. In another example, one or more polymer coatings including a therapeutic agent can be applied to the covering 114 so that the therapeutic agent elutes from the polymer coating after the extruded stent 110 is implanted in the patient.

The stent 112 may be any variety of implantable prosthetic devices known in the art and capable of carrying a covering. The stent 112 can be any elastic material capable of being elastically compressed to a desired diameter in a contracting die. Typically, the stent 112 can be made of a shape memory metal, such as nitinol. The stent 112 can be formed through various methods. The stent 112 can be laser cut, welded or consist of filaments or fibers, which are wound or braided together in order to form a continuous structure. The cross section of the stent elements 116 can be circular, ellipsoidal, rectangular, hexagonal, square, polygonal, or of other cross-sectional shapes as desired. Depending on the material, the stent 112 can be self-expanding, or be expandable with a balloon or some other device.

The covering 114 may be any variety of coatings capable of coating the stent elements 116 and filling the cells 118. The covering 114 is seamless and can be thinner in the cells 118 than on the stent elements 116. In one example, the covering can have a thickness from one half to three thousandths of an inch, typically having a thickness of about one and one half thousandths of an inch in the cells 118 and a thickness of about two thousandths of an inch over the stent elements 116. Typically, the covering 114 can be a polymer, such as polyamides (nylons), polyurethanes, polyesters, combinations, bi-polymers and co-polymers thereof, or the like. The covering 114 can be applied to the stent 112 by extruding the stent 112 through molten polymer to produce a seamless covering. Typically, the covering 114 is impermeable, but the covering 114 can be permeable or perforated to allow flow through some or all of the cells as desired.

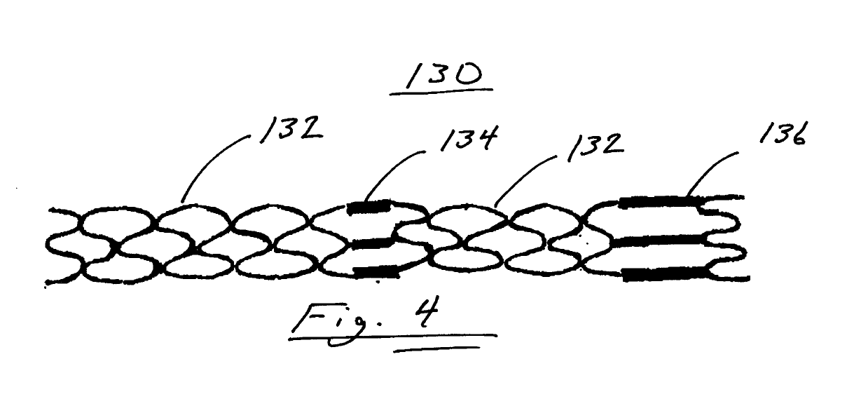

FIG. 4 shows a stent assembly for use in a method of manufacturing a stent with an extruded covering made in accordance with the present invention. The stent assembly 130 comprises stents 132 joined by connectors 134 and having an attachment end 136. Any number of stents 132 can be joined to make the stent assembly 130 as long as the manufacturing equipment can manage the length of the stent assembly. In another embodiment, the stent assembly 130 can be a single stent 132 having an attachment end 136. Typically, at least three connectors 134 evenly spaced around the circumference of the stent assembly 130 are used to provide axial rigidity and assure the stent assembly 130 moves smoothly through the manufacturing system. The attachment end 136 provides means for attaching the stent assembly 130 to a puller for drawing the stent assembly 130 through the extruder and the manufacturing system.

The stent assembly 130 can be manufactured by a number of methods appropriate for the particular materials used. The stent assembly 130 can be laser cut from metal tubing, such as nitinol tubing. In one embodiment, the tubing is at the desired final diameter for the extruded stent when cut. In another embodiment, the tubing is smaller than the desired final diameter for the extruded stent when cut, then the cut tubing is expanded and heat set to the desired final diameter.

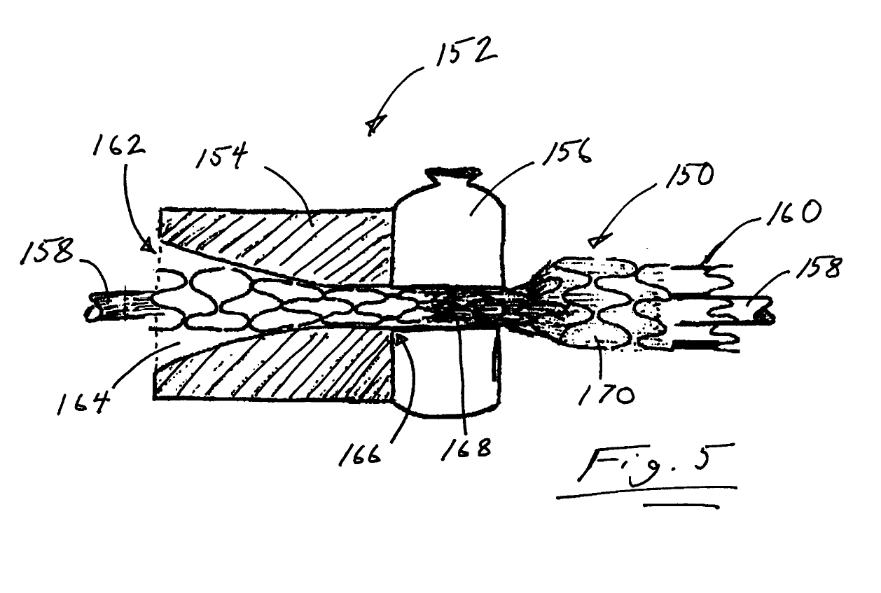

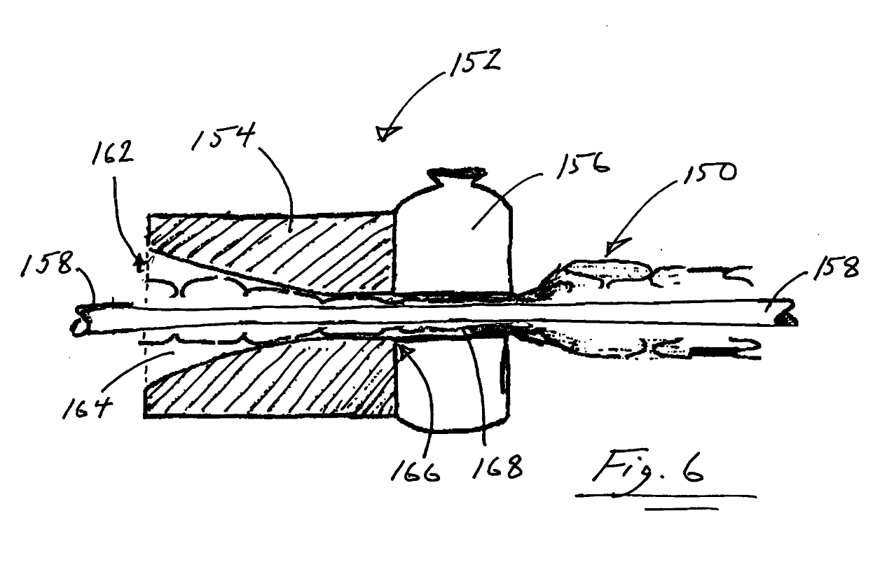

FIGS. 5-8 show methods of manufacturing a stent with an extruded covering made in accordance with the present invention. A stent assembly is compressed to a reduced diameter, coated inside and out with a polymer, and expanded so that the polymer forms a covering. The stent assembly is then separated into individual extruded stents.

FIGS. 5 & 6, in which like elements share like reference numbers, show a method of manufacturing a stent with an extruded covering. The front portion of the manufacturing system has been cut away in FIG. 5 to expose the path of the stent assembly during manufacturing. The stent assembly and the front portion of the manufacturing system have been cut away in FIG. 6 to expose the core mandrel.

A stent assembly 150 is drawn through a manufacturing system 152 comprising a contracting die 154 and an extruder 156. A core mandrel 158 within the stent assembly 150 helps direct the stent assembly 150 through the manufacturing system 152. The attachment end 160 of the stent assembly 150 is attached to a puller (not shown), which draws the stent assembly 150 through the manufacturing system 152.

The stent assembly 150 enters the contracting die 154 at the contracting die mouth 162 of the contracting die passage 164. The contracting die passage 164 tapers to a smaller diameter at the contracting die exit 166, so that the stent assembly 150 is reduced to a compressed diameter. The compressed diameter of the stent assembly 150 can be any desired fraction of the initial diameter of the stent assembly 150, as long as the deformation is primarily elastic and the cells of the stent assembly 150 are sufficiently open so that the molten polymer in the extruder 156 can cover the inside and outside of the stent assembly 150. Generally, the contracting die 154 compresses the stent assembly 150 so that the diameter of the compressed stent assembly is about 20 to 50 percent of the diameter of the uncompressed stent assembly. For example, a stent with an uncompressed diameter of 36 mm may have a compressed diameter from about 8 mm to about 18 mm.

The compressed stent assembly 150 passes through an extruder passage 168 in the extruder 156. The extruder passage 168 contains molten polymer, which coats the inside, coats the outside, and fills the cells of the stent assembly 150. The molten polymer can be any suitable polymer, such as polyamides (nylons), polyurethanes, polyesters, combinations thereof, or the like. Typical temperatures for the various molten polymers in the extruder are in the 200 to 700 degree Fahrenheit range.

The compressed stent assembly 150 expands back to the stent assembly's initial diameter on leaving the extruder 156, primarily due to the elasticity of the stent assembly 150. As the stent assembly 150 expands, the molten polymer in the cells 170 of the stent assembly 150 stretches and thins. The polymer in the cells 170 and on the stent elements forms the covering of the stent assembly 150. Typically, the covering of the stent assembly 150 is seamless. A thin covering increases stent flexibility. In one example, the covering can have a thickness from one half to three thousandths of an inch, typically having a thickness of about one and one half thousandths of an inch in the cells and having a thickness of about two thousandths of an inch over the stent elements. After the covering of the stent assembly 150 has cooled, the stent assembly 150 can be separated into individual stents by laser or mechanical cutting at the connectors joining the individual stents. The cut ends of the individual stents can be polished as required.

Post treatment can be performed or coatings applied before or after separating the stent assembly 150 into individual stents. The covering can be treated with chemicals or radiation to produce the desired physical characteristics. For example, heat or gamma radiation can be used to cross-link the polymer forming the covering and harden the covering. Polymer coatings containing drugs or therapeutic agents, such as anti-inflammatory or anti-proliferative drugs, can be applied to the stent assembly or individual stents over the covering. Coatings can be applied to the covering by a number of methods, such as spraying, dipping, painting, wiping, rolling, printing, and combinations thereof. For some applications, the polymer coating can be limited to a portion of the stent, such as the outer diameter. In other applications, multiple polymer coating layers are desirable to provide different therapeutic agents in different sequences, e.g., the outermost polymer coating layer provides one therapeutic agent, and then degrades to expose another polymer coating layer with another therapeutic agent. Lubricious coatings, such as hydrophilic or hydrophobic lubricious coatings, can be applied to the stent assembly or individual stents over the covering or polymer coating to reduce friction during stent delivery and implantation. If fluid flow through the stent is desired, such as for an angioplasty stent, the covering can be perforated in some or all of the cells. Those skilled in the art will appreciate that many post treatment techniques can be used and combined to make a stent for a particular application.

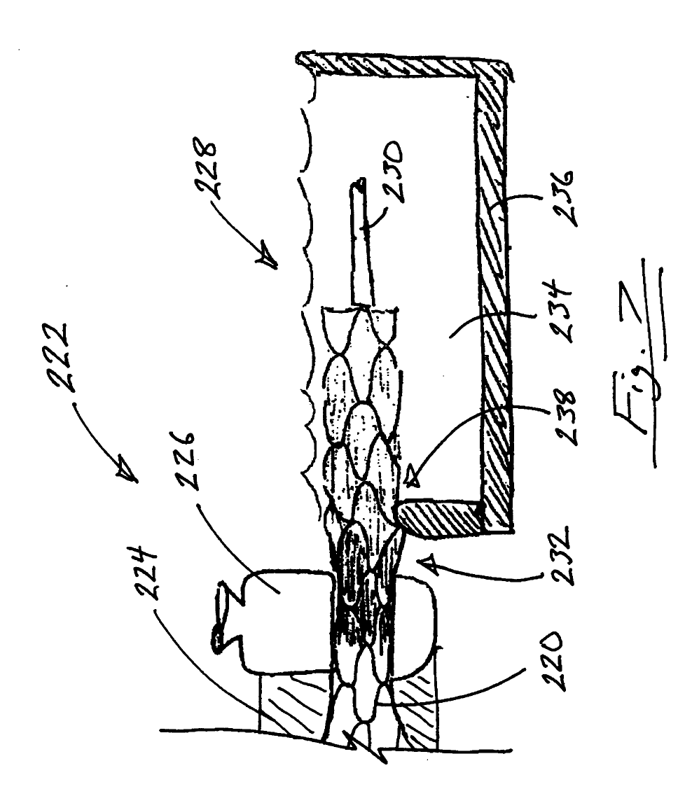

FIG. 7 shows another method of manufacturing a stent with an extruded covering made in accordance with the present invention. A cooling bath is used to control the cooling rate of the stent assembly.

A stent assembly 220 is drawn through a manufacturing system 222 comprising a contracting die 224, extruder 226, and cooling bath 228. A core mandrel 230 within the stent assembly 220 helps direct the stent assembly 220 through the manufacturing system 222. The stent assembly 220 is attached to a puller (not shown), which draws the stent assembly 220 through the manufacturing system 222.

The stent assembly 220 is compressed by the contracting die 224, and coated inside and out with molten polymer in the extruder 226. The stent assembly 220 expands in the gap 232 between the extruder 226 and the cooling bath 228. In one embodiment, the gap 232 can be about 1 to 10 mm. In other embodiments, the gap 232 can be omitted or can be a different width as appropriate for the particular polymer used.

On leaving the gap 232, the stent assembly 220 enters the cooling bath 228. The cooling bath 228 comprises a cooling fluid 234 and a container 236 including a bath entrance 238. The bath entrance 238 can be a notch in the upper edge of the container 236, so that the stent assembly 220 draws the cooling fluid 234 back into the container 236 as the stent assembly 220 enters the cooling bath 228. The cooling fluid 234 can be any cooling fluid compatible with the covering of the stent assembly 220. In one embodiment, the cooling fluid is cooling water. The temperature of the cooling fluid 234 can be set to quickly or gradually cool the covering of the stent assembly 220, as desired for a particular polymer. Once the covering of the stent assembly 220 has cooled, the stent assembly 220 can be separated into individual stents. Post treatment can be performed and coatings can be applied to the stent assembly 220 or the individual stents.

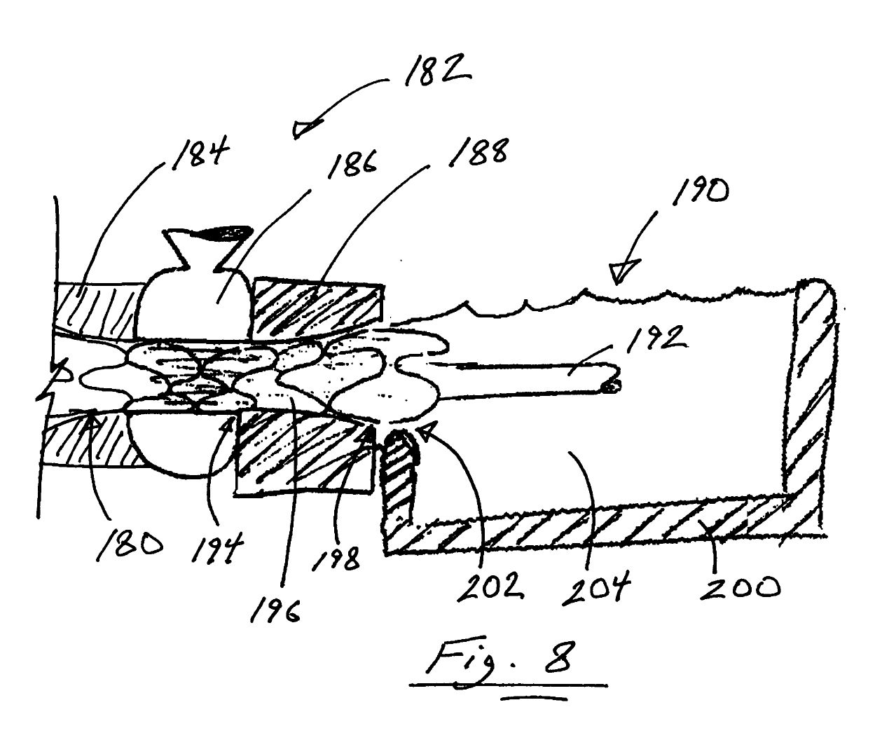

FIG. 8 shows another method of manufacturing a stent with an extruded covering made in accordance with the present invention. An expansion die is used to control the expansion of the stent assembly and a cooling bath used to control the cooling rate of the stent assembly.

A stent assembly 180 is drawn through a manufacturing system 182 comprising a contracting die 184, extruder 186, expansion die 188, and cooling bath 190. A core mandrel 192 within the stent assembly 180 helps direct the stent assembly 180 through the manufacturing system 182. The stent assembly 180 is attached to a puller (not shown), which draws the stent assembly 180 through the manufacturing system 182.

The stent assembly 180 is compressed by the contracting die 184, and coated inside and out with molten polymer in the extruder 186. Rather than letting the stent assembly 180 expand freely on leaving the extruder 186, the stent assembly 180 enters the expansion die mouth 194 and expands gradually through the expansion die passage 196 to the expansion die exit 198. The controlled expansion of the stent assembly 180 in the expansion die 188 avoids tearing of the covering as the molten polymer in the cells of the stent assembly 180 stretches and thins. Controlled expansion may be required for certain polymers. In one embodiment, the diameter of the expansion die exit 198 is the desired final diameter of the stent. In another embodiment, the diameter of the expansion die exit 198 is less than the desired final diameter of the stent and the stent assembly 180 expands further on leaving the expansion die exit 198.

On leaving the expansion die 188, the stent assembly 180 enters the cooling bath 190. The cooling bath 190 comprises a cooling fluid 204 and a container 200 including a bath entrance 202. The bath entrance 202 can be a notch in the upper edge of the container 200, so that the stent assembly 180 draws the cooling fluid 204 back into the container 200 as the stent assembly 180 enters the cooling bath 190. The cooling fluid 204 can be any cooling fluid compatible with the covering of the stent assembly 180. In one embodiment, the cooling fluid is cooling water. The temperature of the cooling fluid 204 can be set to quickly or gradually cool the covering of the stent assembly 180, as desired for a particular polymer. In another embodiment, the cooling bath 190 can be omitted so that the stent assembly 180 enters open air on leaving the expansion die 188. Once the covering of the stent assembly 180 has cooled, the stent assembly 180 can be separated into individual stents. Post treatment can be performed and coatings can be applied to the stent assembly 180 or the individual stents.

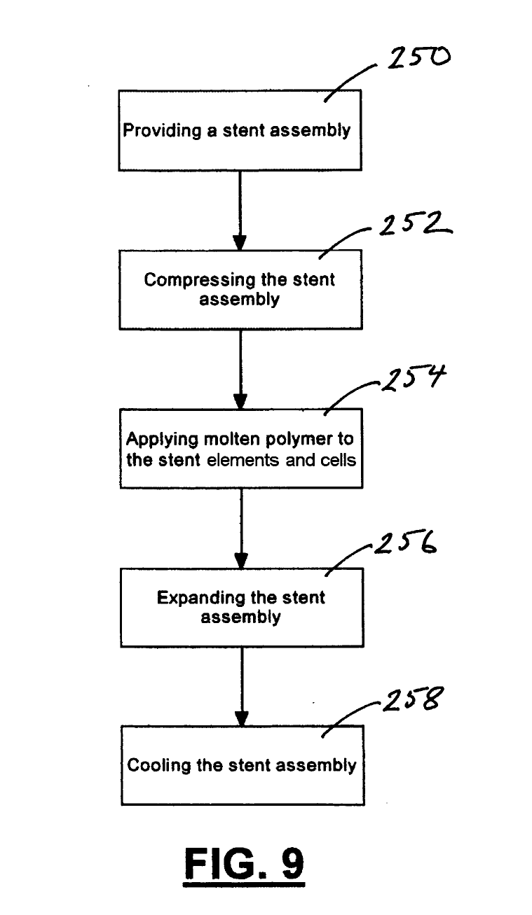

FIG. 9 shows a flow chart of a method of manufacturing a stent with an extruded covering made in accordance with the present invention. At 250, a stent assembly having stent elements forming cells is provided. The stent assembly is compressed radially at 252, such as by compressing in a contracting die. Molten polymer is applied to the stent elements and cells of the stent assembly 254, such as by applying molten polymer with an extruder. The stent assembly expands radially to form a covering 256 and cools 258. In one embodiment, the stent assembly expands freely back to the initial diameter due to the elasticity of the stent assembly. In another embodiment, the stent assembly expands at a controlled expansion rate, such as by expanding the stent assembly within an expansion die. In another embodiment, the stent assembly cools at a controlled cooling rate, such as by cooling in a cooling bath. In another embodiment, the stent assembly expands at a controlled expansion rate and then cools at a controlled cooling rate. Once the stent assembly has cooled, the stent assembly can be separated into individual stents. Post treatment can be performed and coatings can be applied to the stent assembly or the individual stents. Those skilled in the art will appreciate that the methods of manufacture can be varied for the materials used and the results desired.

It is important to note that FIGS. 1-9 illustrate specific applications and embodiments of the present invention, and is not intended to limit the scope of the present disclosure or claims to that which is presented therein. Upon reading the specification and reviewing the drawings hereof, it will become immediately obvious to those skilled in the art that myriad other embodiments of the present invention are possible, and that such embodiments are contemplated and fall within the scope of the presently claimed invention.

While the embodiments of the invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

1. A stent delivery system comprising:
a catheter 102; and
an extruded stent 104 disposed on the catheter 102, the extruded stent 104 having a seamless covering.

2. The stent delivery system of claim 1 wherein the extruded stent 104 is selected from the group consisting of self expanding stents and balloon expandable stents.

3. The stent delivery system of claim 1 further comprising a sheath 106 slidably disposed about the extruded stent 104.

4. The stent delivery system of claim 1 further comprising a balloon operably attached to the catheter 102, the extruded stent 104 being disposed on the balloon.

5. The stent delivery system of claim 4 further comprising a sheath 106 slidably disposed about the extruded stent 104.

6. An extruded stent comprising:
a stent 112, the stent 112 having stent elements 116 forming cells 118;
a covering 114 disposed on the stent 112;
wherein the covering 114 encloses the stent elements 116 and the cells 118, and the covering 114 is seamless.

7. The extruded stent of claim 6 wherein the stent 112 is selected from the group consisting of self expanding stents and balloon expandable stents.

8. The extruded stent of claim 6 wherein the stent 112 is made of a material selected from the group consisting of shape memory metal and nitinol.

9. The extruded stent of claim 6 wherein the covering 114 is a polymer.

10. The extruded stent of claim 9 wherein the polymer is selected from the group consisting of polyamides, nylons, polyurethanes, polyesters, and combinations, bi-polymers and co-polymers thereof.

11. The extruded stent of claim 6 wherein the covering 114 is one half to three thousandths of an inch thick.

12. The extruded stent of claim 6 wherein the covering 114 enclosing the stent elements 116 is thicker than the covering 114 enclosing the cells 118.

13. The extruded stent of claim 6 further comprising a coating containing a therapeutic agent disposed on the covering 114.

14. The extruded stent of claim 6 further comprising a lubricious coating disposed on the covering 114.

15. A method for producing a stent with an extruded covering comprising:
providing a stent assembly, the stent assembly having stent elements forming cells 250;
compressing the stent assembly radially 252;
applying molten polymer to the stent elements and the cells 254;
expanding the stent assembly radially to form a covering 252; and
cooling the stent assembly 254.

16. The method of claim 15 wherein the stent assembly comprises a plurality of stents joined by connectors.

17. The method of claim 15 wherein compressing the stent assembly radially 252 comprises drawing the stent assembly through a contracting die.

18. The method of claim 15 wherein compressing the stent assembly radially 252 comprises compressing the stent assembly radially so that the diameter of the compressed stent assembly is about 20 to 50 percent of the diameter of the uncompressed stent assembly.

19. The method of claim 15 wherein applying molten polymer to the stent elements and the cells 254 comprises drawing the stent assembly through an extruder providing the molten polymer.

20. The method of claim 15 wherein expanding the stent assembly radially to form a covering 252 comprises allowing the stent assembly to expand freely.

21. The method of claim 15 wherein expanding the stent assembly radially to form a covering 252 comprises expanding the stent assembly at a controlled expansion rate.

22. The method of claim 15 wherein expanding the stent assembly radially to form a covering 252 comprises drawing the stent assembly through an expansion die.

23. The method of claim 15 wherein cooling the stent assembly 254 comprises cooling the stent assembly in a cooling bath.

24. The method of claim 15 further comprising separating the stent assembly into individual stents.

25. The method of claim 15 further comprising post treating the covering.

26. The method of claim 25 wherein the post treatment is selected from the group consisting of heat treatment, radiation treatment, and chemical treatment.

27. The method of claim 15 further comprising applying a coating to the covering.

28. The method of claim 27 wherein the coating is applied by a method selected from the group consisting of spraying, dipping, painting, wiping, rolling, printing, and combinations thereof.

29. The method of claim 27 wherein the coating is selected from the group consisting of coatings containing therapeutic agents and lubricious coatings.

30. A system for producing a stent with an extruded covering from a stent assembly, the stent assembly having stent elements forming cells comprising:
means for compressing the stent assembly radially;
means for applying molten polymer to the stent elements and the cells; and
means for expanding the stent assembly radially to form a covering.

31. The system of claim 30 further comprising means for cooling the stent assembly.

32. The system of claim 30 further comprising means for separating the stent assembly into individual stents.

33. The system of claim 30 further comprising means for post treating the covering.

34. The system of claim 30 further comprising means for applying a coating to the covering.
